(19) United States
(12) Patent Application Publication
(10) Pub. No.: US 2026/0207960 A1
(43) Pub. Date: Jul. 23, 2026

(54) Portable Red Light Emitting System and Device

(71) Applicant: Magna Wave Inc., Louisville, KY (US)

(72) Inventors: Cameron O'Nan, Louisville, KY (US); Alane Paulley, Louisville, KY (US)

(21) Appl. No.: 19/452,623

(22) Filed: Jan. 19, 2026

Related U.S. Application Data
(60) Provisional application No. 63/747,184, filed on Jan. 20, 2025.

(51) Int. Cl. A61N 5/06; A61B 17/00

(52) U.S. Cl. CPC A61N 5/06; A61B 2017/00734; A61N 2005/0626; A61N 2005/0652; A61N 2005/0663

(57) ABSTRACT

The novel inclusion of a PEMF shield and integrated battery pack into a red light emitting device and system gives rise to a portable, hand-held, red light emitting system capable of being used simultaneously with and in direct proximity to a PEMF device creating the potential for users to obtain the positive health and wellness benefits of noninvasive red light and PEMF therapies. The lightweight novel red light emitting system does not require use of a wall outlet as a power source or a fan for cooling.

### CROSS-REFERENCE TO RELATED APPLICATIONS

This application claims the benefit of U.S. Provisional Application No. 63/747,184, filed on Jan. 20, 2025, the disclosures of which are incorporated herein by reference.

### FIELD OF THE DISCLOSURE

The present invention relates to a portable, handheld red light emitting device that may be used in conjunction with other health improvement devices, such as a pulsed electromagnetic field machine, to achieve multiple health, beauty, and wellness objectives.

### DISCUSSION OF ART

For decades, light therapy, and specifically, red light therapy, has been used by humans to treat various ailments and improve health and appearance. Among the noted benefits of red light therapy include the improved appearance of skin, treatment of dermatitis, pain management, and treatment of arthritis. Other health and beauty benefits of light therapy and red light therapy are well-known in the art.

The effectiveness of prior art light emitting devices at achieving health and wellness benefits depends on the selected wavelengths of light and output power utilized by the device. The wavelengths of light most often associated with healing and beneficial properties include wavelengths between the visible light spectrum and infrared zone, typically within the range of 605 nm and 970 nm. Accordingly, light therapy devices often incorporate light-emitting diodes (LEDs) operating within said range of wavelengths.

The output power is the amount of energy a device or system produces, typically measured in watts. In addition to total output power, optical output power is the amount of power emitted from a light source. A device may consume 15000 mW electrical power, but if it is inefficient, produce only 5000 mW of optical output power. Prior art light emitting devices typically require a dedicated outlet capable of delivering high wattage power. Even those prior art light emitting devices not producing high wattage power often require a wall outlet and are not capable of being battery operated. Consequently, prior art light emitting devices are typically not portable or efficient. To prevent overheating, these prior art light therapy devices require a fan.

Another drawback of prior art light emitting devices is that, because of their power source, design, and/or configuration, they cannot be used near or in connection with other wellness therapy devices such as pulsed electromagnetic fields (PEMF) devices. Albeit for different reasons, the use of PEMF devices has generated positive health and wellness effects, similar to light therapy. Prior art red light emitting devices, however, may not be used effectively near or in connection with PEMF devices due to magnetic or electrical field interference. Accordingly, a need exists for a portable, battery-operated, red light emitting device and system capable of being used near, in conjunction with, or simultaneously with a PEMF device.

As set forth below, the inventors have developed such device and systems.

### SUMMARY OF THE INVENTION

The present invention describes a novel portable device for emitting red light and a system for improving wellness via simultaneous use of the novel device and a PEMF device. The present invention overcomes the existing shortcomings of prior art systems and devices by providing a portable, hand-held, battery-operated device for emitting red light capable of being used in conjunction with or in proximity to PEMF devices. This novel system achieves the same or increased output power as prior art light emitting systems and devices and may provide the same beneficial and positive health effects as said prior art red light emitting devices with the additional benefit of having the capability of being used simultaneously with PEMF devices for improved wellness.

Importantly, the system comprises a PEMF shield, an integrated battery pack, a plurality of LEDs; an outer enclosure shell; and an external user control interface. In a preferred embodiment of the invention, the device and system comprise an optical output power of 9000 milliwatts (mW). In a preferred embodiment, the integrated battery pack comprises a four (4) cell 16.8V lithium-ion battery with connector and an outer housing shell. In some embodiments, the PEMF shield comprises a bent sheet metal 5052 aluminum part tightly forming around a control printed circuit board (PCB) to shield it from the high energy pulses from a PEMF machine to improve reliability of device operation. In a preferred embodiment of the system and device, the plurality of LEDs comprises an array of fourteen (14) LEDs arranged in two (2) rows on an output LED PCB, wherein eight (8) of said LEDs operate at 660 nm and six (6) of said LEDS operate at 810 nm. In a preferred embodiment, the output LED PCB is manufactured with an aluminum core for added heat dissipation. In a preferred embodiment, the external user control panel is connected to the control PCB. The external user control panel further comprises control buttons for turning the power on and off and for selecting between a 30 second or 60 second time period of red light emission. In a preferred embodiment, the system and device weighs about 0.8 lbs. In a preferred embodiment, the red light emitting system and device does not comprise a fan.

The present invention is intended to emit red light while not connected to an external wired power source allowing users to obtain the positive health and wellness benefits of red light therapy without the need for a wall outlet as a power source.

The present invention further includes systems for improving wellness comprising use of the novel portable red light emitting device simultaneous with or in proximity to use of a PEMF device, both of which are used externally as non-invasive therapy options for ailments such as arthritis. Instead of directing light within certain wavelengths at specific areas of the body over short periods of time, PEMF devices apply intermittent current pulse-generated magnetic field pulses over a short time to specific areas of the body. Use of PEMF devices has generated positive health and wellness effects, similar to light therapy. For example, among the noted benefits of using PEMF devices include pain management, treatment of arthritis, improved bone health and healing, and treatment of neurological and dental conditions. Target areas for external application of the novel portable red light emitting device and PEMF devices include various body parts of humans and animals. For example, therapy using red light emissions and PEMF devices may be targeted to areas such as joints, the back and spine, soft tissues and muscles, head and neck, and feet and ankles.

In the novel systems disclosed herein, the portable red light emitting device is used in proximity to, and simultaneously with, a PEMF device or attachment. In such systems, the portable red light emitting device may be used at any distance from the PEMF device without interfering with and/or reducing the functionality or performance of said red light emitting capabilities. The present invention is intended for use on humans and/or animals.

Other features and advantages of the present invention will become apparent from the following more detailed description, taken in conjunction with the accompanying figures, which illustrate, by way of example, the principles of the invention.

### BRIEF DESCRIPTION OF THE FIGURES

FIG. 1 depicts a schematic view of the red light emitting system (1), including the control PCB (2), PEMF shield (3), integrated battery pack (4), output LED PCB (5), plurality of LEDs (8), protective window (6), and outer enclosure shell (7).

FIG. 2 depicts a schematic view of the outward-facing surface of the red light emitting system (1) including the output LED PCB (5), plurality of LEDs (9), and outer enclosure shell (7).

FIG. 3 depicts a schematic view of the red light emitting system (1) in use.

FIG. 4 depicts a schematic view of the output LED PCB (5).

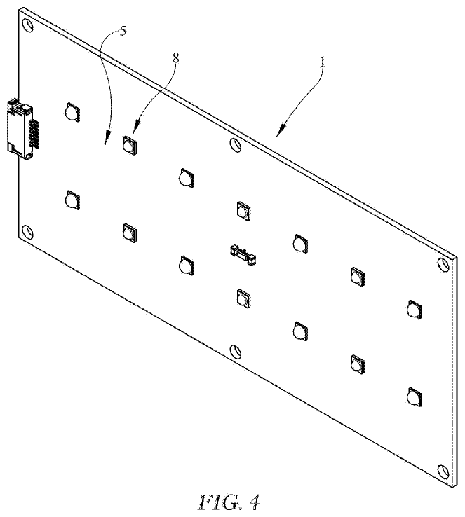

FIG. 5 depicts a schematic view of the control PCB (2).

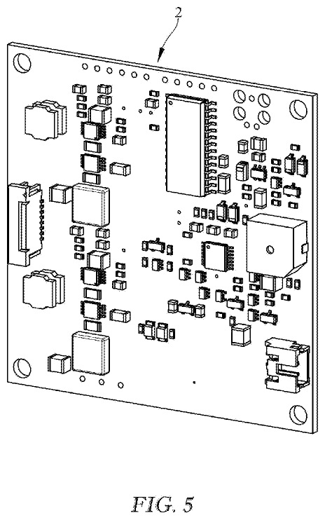

FIG. 6 depicts a schematic view of the PEMF shield (3).

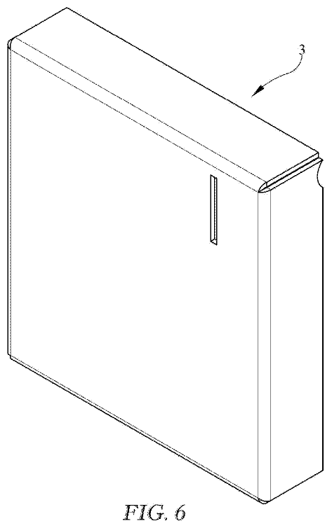

FIG. 7 depicts a schematic view of the integrated battery pack (4).

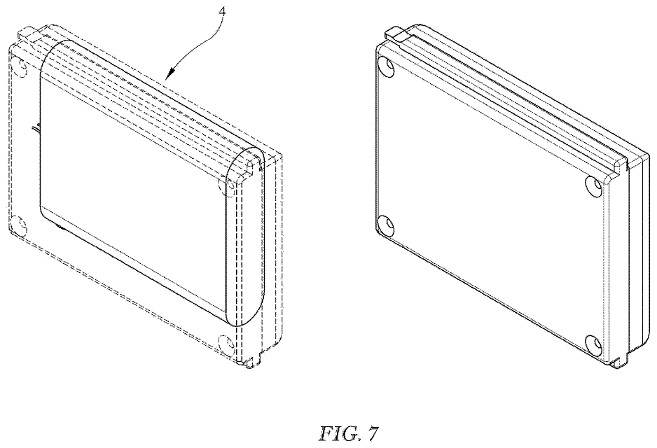

FIG. 8 depicts a view of the outward-facing surface of the system (1).

FIG. 9 depicts a view of the user-facing surface of the system (1).

FIG. 10 depicts a schematic view of the red light emitting system (1)

FIG. 11 depicts a schematic view of the control PCB (2) and LED PCB (5)

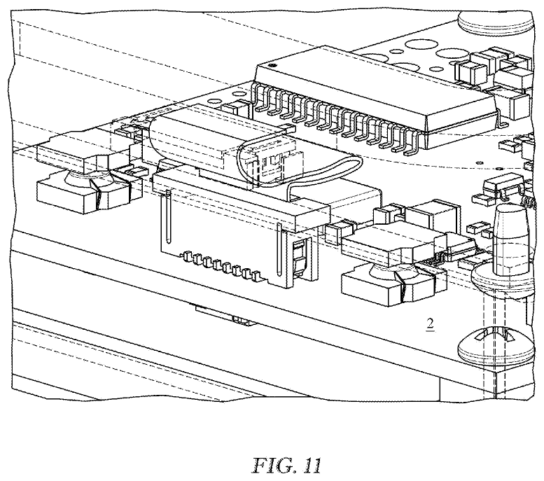

FIG. 12 depicts a side view of the red light emitting system (1) showing the control PCB (2), PEMF shield (3), LED PCB (5) and ribbon cable (5a).

### DETAILED DESCRIPTION OF THE INVENTION

The following discussion addresses a number of embodiments and applications of the present invention. Reference is made to the accompanying figures that form a part hereof and show by way of illustration specific embodiments in which the invention may be practiced. It is to be understood that other embodiments may be utilized and changes may be made without departing from the scope of the present invention.

Various inventive features are described below that can be used independently of one another or in combination with other features. However, any single inventive feature may not address the problems discussed above or only address one of the problems discussed above.

As used herein, the singular forms “a”, “an” and “the” include plural referents unless the context clearly dictates otherwise. “And” as used herein is interchangeably used with “or” unless expressly stated otherwise. As used herein, the term ‘about” means +/−5% of the recited parameter. All embodiments of any aspect of the invention can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “wherein”, “whereas”, “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

The present invention provides for a portable red light emitting system capable of being used in conjunction with and in proximity to a PEMF device without experiencing any reduction in the functionality or performance of the red light emitting capabilities of the present invention. The present invention may be used directly adjacent to a PEMF device without any distance limitation or minimum distance requirement. As described herein, the present invention provides for a portable, battery-operated red light emitting system that does not require a fan for cooling or an outlet as a power source. Accordingly, the present invention provides for a low-weight device and system that an individual user can hold in one hand and apply to the desired target area.

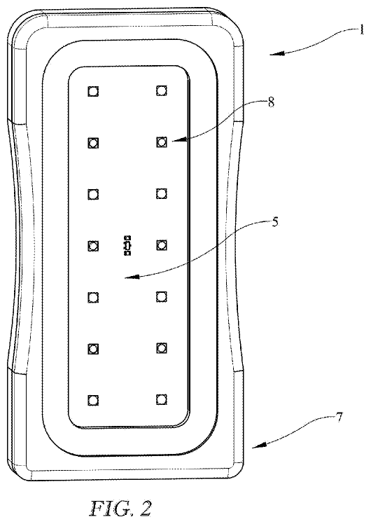

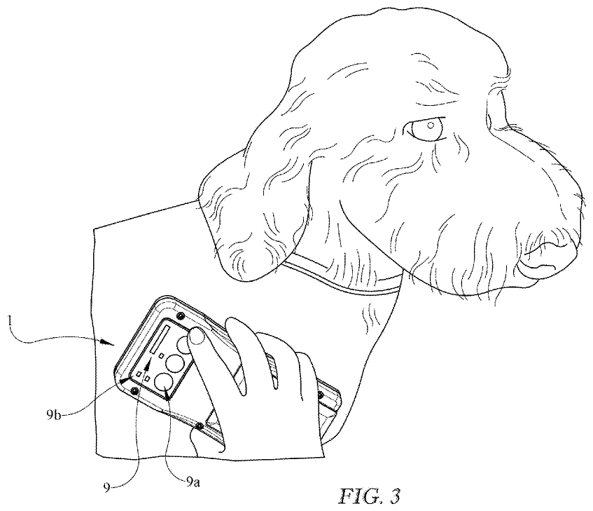

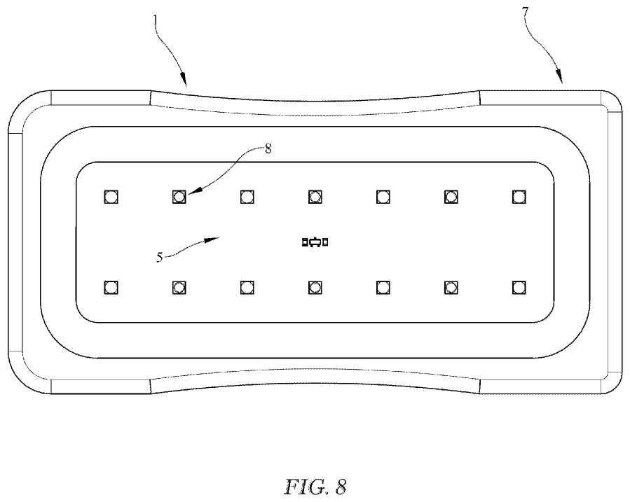

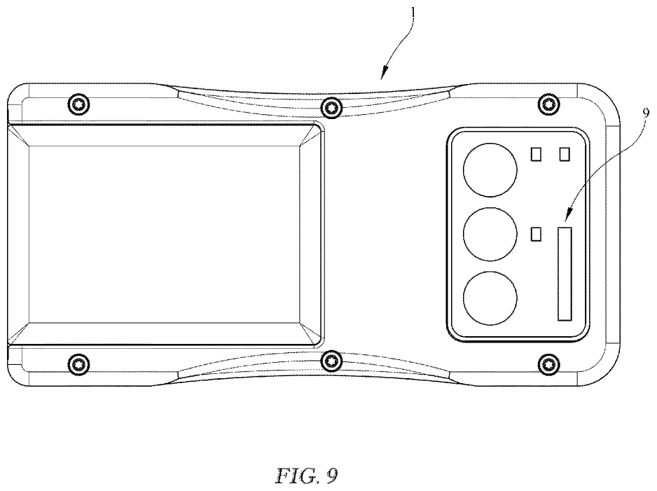

As generally shown in the Figures, the portable red light emitting system comprises an electronic device. Said device includes a housing having a generally rectangular prismatic shape with rounded corners, the housing defining a user-facing surface (see FIG. 9), an outward-facing surface opposite the user-facing surface (see FIG. 8), and a peripheral side surface extending between the user-facing and outward-facing surfaces. As shown in FIGS. 3 and 9, the user-facing surface includes a display configured to present a user control interface (9), wherein a user may control the power and function of the system (9a, 9b). As shown in FIGS. 2 and 8, the outward-facing surface includes a translucent protective window (6) coupled to an output LED PCB (5) for emitting light to a target area. The peripheral side surface includes four lateral side portions corresponding to first and second long sides and first and second short sides (see FIG. 2).

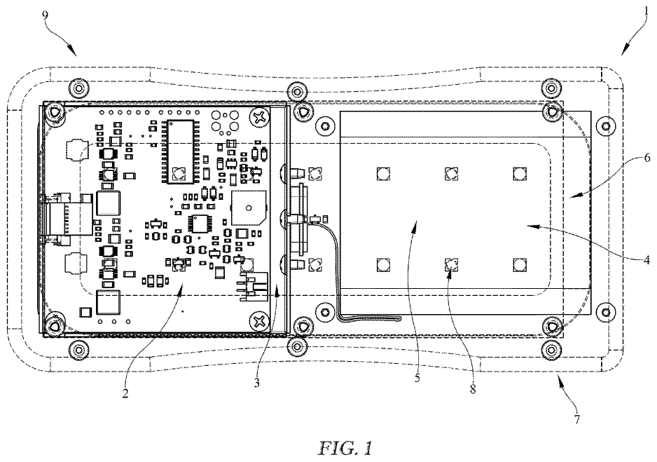

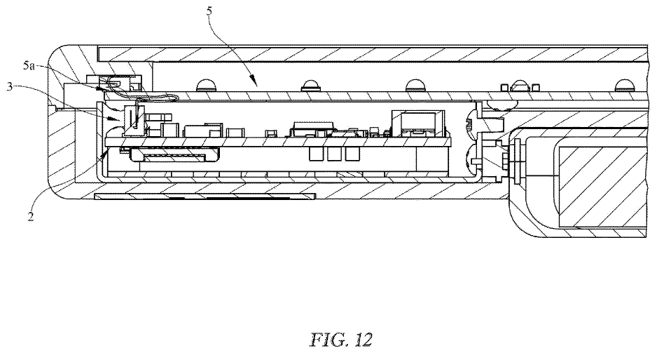

As set forth in FIG. 1, in a preferred embodiment, the inner housing of the red light emitting device comprises a central control panel consisting of a control PCB (2), a PEMF shield (3), and an integrated battery pack (4). In a preferred embodiment, the control PCB (2) is positioned adjacent to the integrated battery pack (4) in a substantially horizonal arrangement on a first horizontal plane. As shown in FIG. 12, the control PCB (2) and the PEMF shield (3) are substantially aligned along the thickness axis such that the control PCB (2) is directly above the PEMF shield (3). The output LED PCB (5) is disposed between the outward-facing surface and the first horizontal plane comprising the control PCB (2) and integrated battery pack (4).

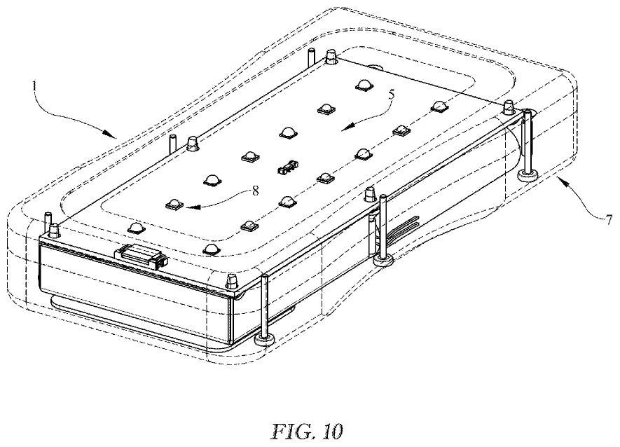

As shown in FIGS. 1, 10, and 12, in a preferred embodiment, the output LED PCB (5) comprises a plurality of LEDs (8) configured in two rows. In a preferred embodiment the output LED PCB is about 60×150 mm; the control PCB is about 80×80 mm; the PEMF shield is about 90×90 mm; and the integrated battery pack is about 70×90 mm. Other embodiments may comprise different configurations or arrangements of the control PCB, integrated battery pack, PEMF shield, and output LED PCB.

Turning to the control PCB (2), it comprises drive circuitry including LED driver circuits, a microcontroller, and hardware safety backup circuits to monitor and control LED output. In a preferred embodiment the control PCB contains an LED driver circuit for each wavelength of LEDs used on the output LED PCB. In a preferred embodiment, the control PCB further comprises a microcontroller for taking user input from the control buttons on the user control interface and actuating the LED output using firmware written in C programming language. In a preferred embodiment, the control PCB further comprises hardware backups to the firmware as an added safety precaution. In a preferred embodiment the control PCB is made of FR4 fiberglass. In other embodiments, the control PCB may be made of any non-conductive materials, including plastic, glass-fiber, or resin components and is primarily intended to direct the flow of currents in the red-light emitting device. Methods for making and configuring PCBs are known in the art and may be employed in connection with the present invention. As shown in FIGS. 1 and 3, in the preferred embodiment, the control PCB is connected to a corresponding user control interface (9) that allows the user to control red light emissions and connected to the output LED PCB via a ribbon cable.

In a preferred embodiment, the user control interface (9) comprises a plurality of control buttons (9a) including a button for the user to turn the power “on” or “off.” In a preferred embodiment, the user control interface (9) further comprises a plurality of indicator lights (9b) including an indicator light when the power is turned “on.” In a preferred embodiment, the user control interface (9) further comprises a second button allowing a user to select to start or pause red light emissions when the power is turned “on.” In a preferred embodiment, the user control interface further comprises a third button to control the duration of red light emissions when the power is turned “on.” In a preferred embodiment, the third button allows the user to select between a duration of 30 seconds or 60 seconds. In a preferred embodiment, the user control interface (9) further comprises a second indicator light when the duration of 30 seconds is selected and a third indicator light when the duration of 60 seconds is selected. In other embodiments, the user control interface may comprise additional control buttons corresponding to different time periods and/or intensities of preferred red light emissions.

In a preferred embodiment, the control PCB is positioned directly above the PEMF shield (3). The PEMF shield (3) is made of bent sheet metal 5052 aluminum part. The PEMF shield protects the control PCB from high strength electric and magnetic fields by acting as a barrier to absorb and reflect electromagnetic radiation. Due to its electrical conductivity, the PEMF shield reduces the field strength that reaches the control PCB. The novel inclusion of the PEMF shield in the portable red light emitting device and system described herein allows the device to be used in conjunction with or proximity to a PEMF device without said PEMF device interfering with the functionality or performance of the red-light emissions.

The capability of the portable red light emitting device that is the present invention to be used “in conjunction with” or “in proximity to” a PEMF device means the present invention is resistant to high strength PEMF even when placed in direct contact with a high-intensity, professional grade therapeutic PEMF device. PEMF devices operate at frequencies ranging from 1 hertz (Hz) to 1000 Hz and typically use intensities between 1 gauss (G) and 100 G, although some PEMF devices can deliver up to 8,000 G. The inventors tested the compatibility of the portable red light emitting device with a PEMF device by using a PEMF device pulsing at its highest setting on a surface while simultaneously moving the portable red light emitting device across the same surface in every orientation with its LED output active. This was performed for ten minutes of continuous use of both the PEMF device and the portable red light emitting device that is the subject of the present invention, with the present invention behaving as expected throughout the testing period, indicating the portable red light emitting device is resistant to PEMF and can be used at any distance from a PEMF device. Specifically, the portable red light emitting device that is the subject of the present invention was placed on top of an active MagnaWave Julian PEMF machine with a paddle accessory, which has twenty intensity settings and is capable of delivering 8000 Gauss. Said experiments further demonstrated that the present invention may be used simultaneously with a PEMF machine with no distance limitations. The simultaneous use of the present invention and PEMF machine may increase the potential for achieving positive wellness benefits such as improved skin appearance, reduction in inflammation, and pain management.

Adjacent to the control PCB (3) within the central control panel is the integrated battery pack (4). In a preferred embodiment, the integrated battery pack comprises a 4 cell 16.8V lithium-ion battery with connector and outer housing shell. A main feature of the novel system and device described herein is the use of energy dense lithium ion batteries. Energy dense lithium batteries allow for a practical amount of run time for their weight whereas other battery chemistries may not allow the energy density for practical handheld use. In a preferred embodiment, the battery comprising the integrated battery pack is chargeable. In a preferred embodiment, the battery pack has one (1) hour continuous run time. In a preferred embodiment, each of the integrated battery pack charges fully in about 2.5 hours. In a preferred embodiment of the present invention, actual optical output measurement is 9000 mW.

As shown in FIGS. 1 and 12 the output LED PCB is positioned between the outward-facing surface and the portion of the control panel comprising the control PCB and integrated battery. The control PCB (2) is connected with a ribbon cable (5a) to the output LED PCB (5). The output LED PCB (5) is generally rectangular and comprises a plurality of LEDs (8) for producing red light emissions. See FIGS. 1-3. In a preferred embodiment the generally rectangular output LED PCB (5) has a length of about 150 mm and width of about 60 mm. In a preferred embodiment, the output LED PCB (5) comprises an array of 14 LEDs arranged in two horizontal rows, wherein each horizontal row includes 7 LEDs. In the preferred embodiment, the plurality of LEDs (8) operate within one or more of the following wavelengths: 8 LEDs at 660 nm and 6 LEDs at 810 nm. The output LED PCB further comprises a sensor to monitor surface temperature on the device for safety and to reduce the potential of damage.

In other embodiments, the plurality of LEDs may comprise different arrangements of LEDS involving a different quantity of LEDs arranged in a variety of rows, shapes, and arrays. In other embodiments, the plurality of LEDs may operate at other wavelengths between the visible light spectrum and infrared light zone. In the preferred embodiment, the output LED PCB comprises an aluminum as opposed to fiberglass to provide additional heat dissipation qualities.

In a preferred embodiment, a clear, translucent protective window (6) covers the output LED PCB (5). In a preferred embodiment, the protective window (6) is generally rectangular and is capable of covering the entirety of the LED PCB (5). In a preferred embodiment, the generally rectangular protective window has a length of about 150 mm and a width of about 65 mm. The protective window may be made of acrylic.

As shown in FIGS. 1-3, an outer enclosure shell (7) encloses the housing for the device. The outer enclosure shell (7) is a generally rectangular box having an outward-facing side corresponding to the outward-facing surface of the device and a user-facing side corresponding to the outward-facing surface of the device. As shown in FIGS. 1 and 2, the outward-facing side of outer enclosure shell comprises a first opening sufficient to display the plurality of LEDs and the protective window. As shown in FIG. 3, the user-facing side of the outer enclosure shell comprises a second opening sufficient to display the user control interface (9). In a preferred embodiment, the outer enclosure shell (7) is made of PA12 nylon sintered powder or similar rigid and material.

The foregoing description of several embodiments of the novel, red light emitting device and system for use in conjunction with or proximity to a PEMF device have been presented for purposes of illustration. It is not intended to be exhaustive or to limit the application to the precise forms disclosed, and obviously, many modifications and variations are possible in light of the above teaching. It is understood that the invention may be applied in ways other than as specifically set forth herein without departing from the scope of the invention.

What is claimed is:

1. An illumination system comprising:
a housing;
a red-light emitter comprising a plurality of LEDs for emitting electromagnetic radiation in a red wavelength band;
a PEMF shield for preventing electrical and magnetic field interference with a PEMF device;
a self-contained power source comprising an integrated battery pack; and
a control PCB coupled to a user control interface for a user to select a preferred duration of red light emissions;
wherein the red-light emitter is operable while not connected to an external wired power source.

2. The system of claim 1:
wherein the red-light emitter, PEMF shield, integrated battery pack, and control PCB are contained in the housing, said housing having a generally rectangular prismatic shape with rounded corners; said housing further defining a user-facing surface, an outward-facing surface opposite the user-facing surface, and a peripheral side surface extending between the user-facing surface and the outward-facing surface;
wherein the user-facing surface includes the user control interface;
wherein the plurality of LEDs comprises an array of fourteen LEDs arranged in two rows on an output LED PCB, said output LED PCB coupled to a translucent protective window on the outward-facing surface;
wherein the control PCB is disposed between the LED PCB and the user-facing surface, said control PCB connected to the output LED PCB via a ribbon cable;
wherein the control PCB and the PEMF shield are substantially aligned along a thickness axis such that the control PCB is directly above the PEMF shield, said PEMF shield being proximal to the user-facing surface and distal to the outward-facing surface; and
wherein the housing is enclosed in an outer enclosure shell.

3. The system of claim 2 wherein the red wavelength band comprises wavelengths from the group of wavelengths consisting of the following: 660 nm and 810 nm.

4. The system of claim 2 wherein the control PCB comprises drive circuitry coupled to the output LED PCB and the integrated battery pack, the drive circuitry configured to power the LED PCB from the integrated battery pack.

5. The system of claim 1 wherein the integrated battery pack is a 4 cell 16.8V lithium-ion battery

6. The system of claim 1 wherein the PEMF shield is made of bent sheet metal 5052 aluminum part.

7. The system of claim 1 whereby the system is used directly adjacent to and simultaneously with a PEMF device.

8. The system of claim 1 comprising an optical output power of 9,000 mW.

9. The system of claim 2 whereby the output LED PCB is manufactured with an aluminum core for added heat dissipation and the system is operable to emit red light without a fan for cooling.

10. The system of claim 1 wherein the user control interface comprises control buttons to select between a 30 second time period and 60 second time period for red light emissions.

11. The system of claim 1 wherein the integrated battery pack comprises a chargeable battery capable of a continuous run time of one hour.

12. The system of claim 1 wherein the system weighs about 0.8 pounds.

13. The system of claim 1, wherein the control PCB further comprises a microcontroller for taking input from the user control interface and actuating output of the plurality of LEDs using firmware written in C programming language.

14. A system for improving wellness including:
a target area comprising a body part;
a portable red light emitting device for externally directing red light emissions to the target area, said device comprising a plurality of LEDs, a control PCB, a PEMF shield, an integrated battery pack, and a user control interface; and
a PEMF device for externally applying a plurality of intermittent current pulse-generated magnetic field pulses to the target area;
wherein the portable redlight emitting device and PEMF device are simultaneously applied to the target area without any requirement for a minimum distance between the portable redlight emitting device and PEMF device; and
wherein the magnetic field pulses of the PEMF device do not interfere with the red light emissions of the portable red light emitting device.

15. The system of claim 11 wherein the plurality of LEDs operate at a wavelength from the group of wavelengths consisting of the following: 660 nm and 810 nm.

16. The system of claim 11 wherein the portable red light emitting device produces an optical output power of 9,000 mW.

17. The system of claim 11 wherein the portable red light emitting device is operable while not connected to an external wired power source.
